(19) United States
(12) Patent Application Publication
(10) Pub. No.: US 2026/0315095 A1
(43) Pub. Date: Oct. 15, 2026

(54) TRANSCATHETER ULTRASOUND DEBRIDEMENT OF FIBROCALCIFIC VALVE AND ANCILLARY MECHANICAL LEAFLET EXPANSION ASSOCIATED WITH A TEMPORARY VALVE PROSTHESIS

(71) Applicant: AORTICLAB SRL, Colleretto Giacosa (TO) (IT)

(72) Inventors: Enrico FERMI, Piacenza (IT); Enrico PASQUINO, Savigny (CH); Francesco BONETTI, Torino (TO) (IT); Franco OSTA, Mombello Monferrato (AL) (IT); Mario DI CECIO, Marcheno (Brescia) (IT)

(21) Appl. No.: 19/484,511

(22) PCT Filed: May 8, 2024
(86) PCT No.: PCT/EP2024/062685

§ 371 Date: Nov. 14, 2025

(30) Foreign Application Priority Data
May 15, 2023 (WO) PCT/EP2023/062890

(51) Int. Cl. A61B 17/32; A61B 17/00; A61F 2/24

(52) U.S. Cl. CPC A61B 17/320068; A61B 17/00234; A61F 2/2418; A61F 2/2436; A61B 2017/00243; A61B 2017/00305; A61B 2017/320069

(57) ABSTRACT

Device for the treatment of aor c stenosis comprising at least one ultrasonic abla on unit and one expandable stent valve.

### FIELD OF INVENTION

This invention relates to systems for the treatment of aortic stenosis due to fibrosis and/or calcification of the valve leaflets, with the aim of improving or restoring an adequate hemodynamic profile of the patient, deferring the implantation of an artificial bioprosthesis, of limited duration, often lower than the patient's life expectancy.

### BACKGROUND

The treatment of aortic valve stenosis is frequently based on the implantation of a valve bioprosthesis using the TAVI procedure or surgical aortic valve replacement.

The use of TAVI has increased markedly in younger patients, despite the lack of data on long-term valve durability.

Another possibility is the balloon aortic valvuloplasty that has been used as an alternative approach in patients who are not fit for surgery, but this is usually tailored to the patient. Balloon aortic valvuloplasty can be associated with poor outcomes, but in specific patients who are haemodynamically unstable it may serve as a bridge to surgery or TAVI, or as a palliative measure where surgery is contraindicated.

Conservative solutions are being studied for the treatment of aortic valve stenosis with the aim to restore, in whole or in part, the pliability of the valvular leaflets, to restore the flow and reduce aortic valvular stenosis. The use of modulated ultrasound seems to represent a promising solution, especially if the ultrasound field has the form of shockwaves, delivered directly to the leaflet of the aortic valve to be treated.

In the patent application WO2020/151995A1 a transcatheter device is described for the treatment of aortic valve stenosis due to calcific dystrophy, through the emission of an impulsive ultrasound field at two different frequencies. The device includes a temporary artificial valve capable of replacing the activity of the patient's native valve during the duration of the treatment.

The solution described, however, has some limitations, in particular:
the availability of a limited number of sizes of devices compared to the great variability of the sizes of the patients' native valves;
the risk that the artificial valve does not always adapt correctly to the native valve due to the many physiological variations present in cases of aortic valve stenosis (risk of paravalvular leakage).

### GENERAL DESCRIPTION OF THE INVENTION

The present invention concerns a device as defined in the claims.

More precisely the invention concerns a device for the treatment of aortic stenosis comprising at least one ultrasonic ablation unit and one expandable stent valve.

Preferably the ablation unit(s) is/are configured to directly act on aortic valve leaflet(s).

Advantageously the expandable stent valve is configured to clamp an aortic valve leaflet between said stent valve and one ablation unit.

The device according to the invention performs the function of treating aortic valve stenosis by combining the action of, preferably low-energy, ultrasound waves with the mechanical stretch action of a stent valve that adapts to the patient's native valve. Ultrasounds allow the debridement of calcified deposits or the modification of the characteristics of the fibrotic leaflets, through “spaces” created in the spongiosa by the effect of cavitation bubbles of dissolved gas, inside the spongiosa-layer. The result is the restoration of pliability and the improvement of blood flow and reduction of the transvalvular pressure gradient.

The gas bubbles, subjected to the shockwaves, under compression and decompression forces, expand and collapse causing mechanical erosion due to the concentrated release of energy and restoring the leaflet pliability. The piezoelectric transducers may advantageously generate two low intensity ultrasound shockwaves, for instance around 3 MHz and 100-200 KHz frequencies, without temperature increase. The combination of two frequencies enhances the cavitation effect and so the efficacy of the treatment.

The invention is also applicable to the treatment of degenerated bioprosthesis.

Specifically, the invention relates to a novel device that uses, preferably low-energy, shock waves emitted via ultrasonic (e.g. piezoceramic) transducers directly on valve leaflets in combination with the stretch action of an expandable stent, equipped with an artificial temporary valve.

This expandable stent valve combines the pushing action on the aortic valve, with the effect of ultrasounds on the leaflets.

### DETAILED DESCRIPTION OF THE INVENTION

The invention will be described in a more detailed manner in the present chapter, with some illustrated examples.

The invention is of course not limited to the examples and alternative embodiments discussed in this chapter.

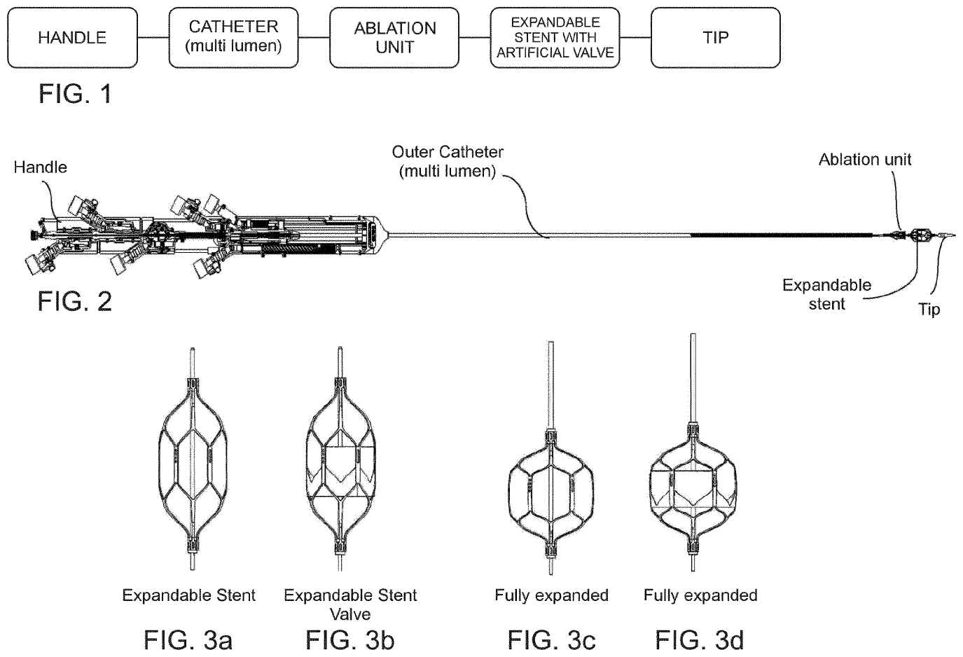

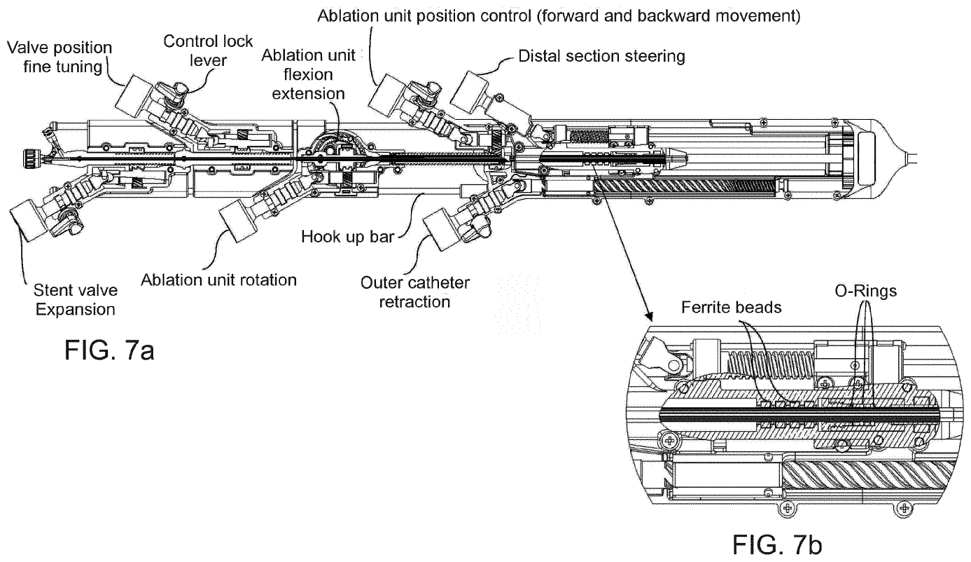

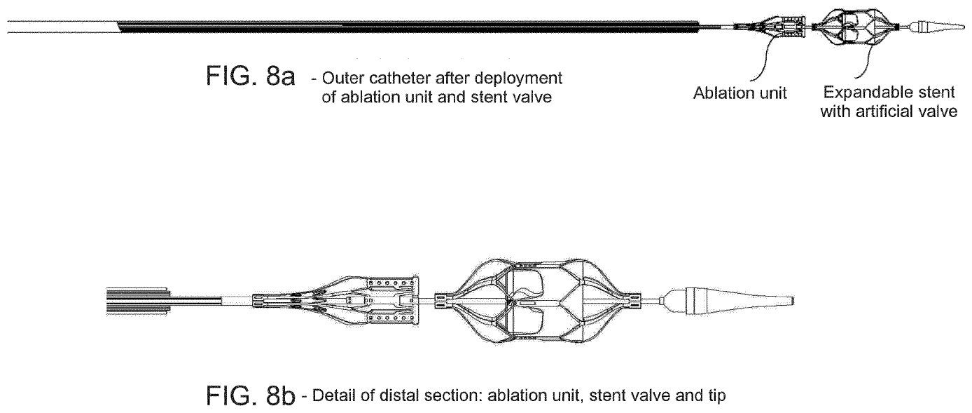

One example of the device according to the invention is illustrated in FIGS. 1 and 2. It includes:
a “deployment” section, i.e. a catheter to reach the heart valve site, through percutaneous access;
a multi lumen catheter that allows the passage of electrical connection wires with the ablation unit(s) and pulling wires to allow the movements of those various elements;
a bi lumen tube;
a temporary artificial valve, preferably made of silicone or tissue, located within an expandable stent (FIGS. 3a, 3b, 3c, 3d, 8b);
a handle with the movement controls of the various elements (FIG. 7a)

According to a preferred embodiment of the invention, the motion controls managed through the handle is achieved as follows:
steering of the distal section of the outer catheter of the device;
retraction of the external sheath of the device to allow the opening of the artificial valve and the release of the ablation units;
flexion and extension of the ablation units;
rotation of the ablation units with 1:1 torque transmission cable mounted on steel tube: this solution can guarantee +/−180° torquability when the device is deployed at the extrados of the aortic arch.
fine positioning of the artificial valve inside the native valve
controlled expansion of the expandable stent.

The impulsive electrical signal may be supplied by a voltage generator capable of supplying signals, preferably at two alternating frequencies of approximately 3 MHz and 100-200KHz.

The ablation units are similar to those described in patent application WO2020/151995A1 and may therefore be based on lead zirconate titanate PZT piezoceramic transducers, mounted on support structures which provide the backing function (ultrasound focusing without back scattering). Other methods besides PZT can be employed to generate shockwave ultrasounds, such as arc discharge in water, according to scientific literature.

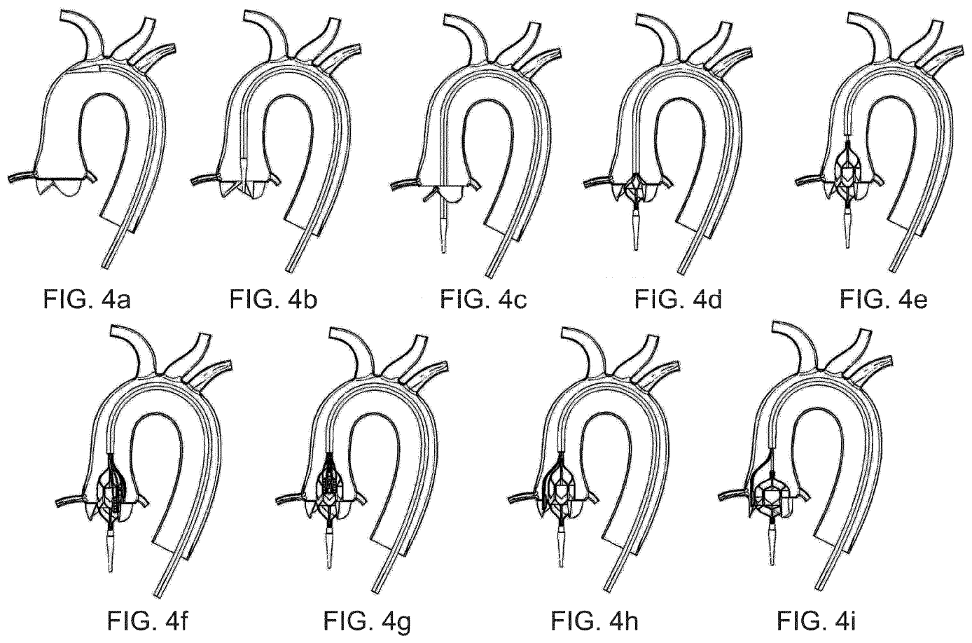

The catheter advances in the vessels of the lower limbs, from the femoral access, along the iliac artery, the abdominal and thoracic aorta, to reach, through the aortic arch, the stenotic aortic valve (FIGS. 4a, 4b).

The hypotube of the device (FIG. 8), allows the use of a guidewire for the correct navigation of the device and the correct positioning of the artificial valve in the native one of the patient.

Once the native valve seat is reached, with the tip passing through and entering the ventricle, (FIG. 4c), the external sheath is retracted (FIG. 4d), with the opening and positioning of the artificial valve (FIG. 4e).

Once the ablation units have been released, they can be controlled, flexed or extended, advanced or retracted, rotated of 360°, in order to position them in the native valve area to be treated (FIGS. 4f, 4g, 4h).

In this way the patient's valve leaflets are clamped between the ablation units and the valved stent (FIG. 4h). During the ultrasound treatment the characteristics of the leaflets change, improving the pliability. The operator can then expand the valved stent, (FIG. 4i), to obtain both an improvement in the adhesion to the native valve, reducing or eliminating the risk of paravalvular leakage, and a stretching action on the leaflets to improve the efficacy of the treatment (combination of active physical energy and mechanical pushing force: the expandable valve stent can stretch the leaflets under the remodelling effect of ultrasound field).

The present invention includes several original features, such as:
the parallelization of the wide set of controls, described previously, to develop a small-sized device;
the expansion of a valved stent, to perform a sort of delicate valvuloplasty under the action of the ultrasound field, which acts on fibrosis and calcified deposits, reducing both;
the artificial valve, inside the expandable stent, that allows the ultrasound treatment to be carried out for a relatively long period, such as tens of minutes, until the desired haemodynamic improvement is obtained;
the system can be repositioned several times during the treatment session, allowing the control of hemodynamic parameters, and being able to treat different leaflets or different portions of the stenotic native valve;
the expandable stent allows it to adhere properly to the walls of the native valve during treatment.

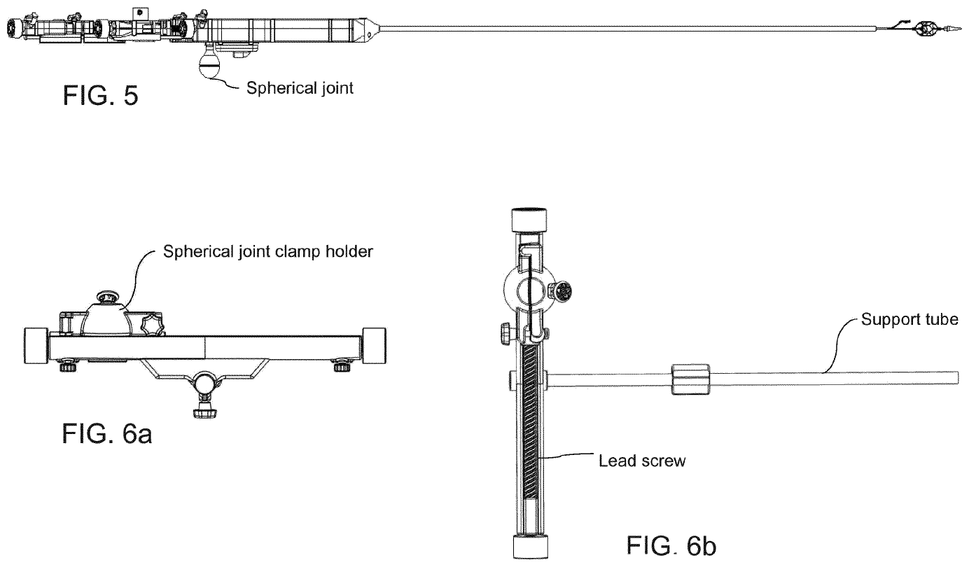

FIG. 1 shows the set of functional modules of the device, represented graphically in FIG. 2 (top view) and FIG. 5 (side view).

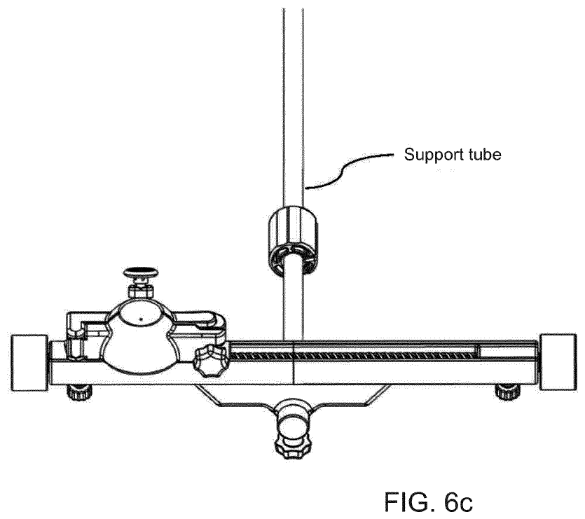

FIGS. 6a, 6b, 6c show different views of the support module for placing the device on the operating table. The spherical joint of the handle can be clamped in the spherical joint clamp holder of the support module. The spherical joint has three rotational degrees of freedom between the two attached components. The device is so free to rotate relative to the support module along all three axes. This solution allows you to orientate the device on the operating bed.

The lead screw in FIG. 6b allows you to move the device along the support module by acting on the knobs at the ends: by turning them, the device moves forward or can be retracted.

The movements of the different “modules” are performed by means of tie wires or moving directly the outer catheter, the multi-lumen or the tubes constituting the sub-units (FIGS. 8, 9, 10, 11,13).

The motion transmission, from the manual rotation of the knob, takes place, in the various sections, through a cardan joint connected to a lead screw (FIG. 14). It is possible to observe this solution in FIGS. 7a, 9, 10, 13.

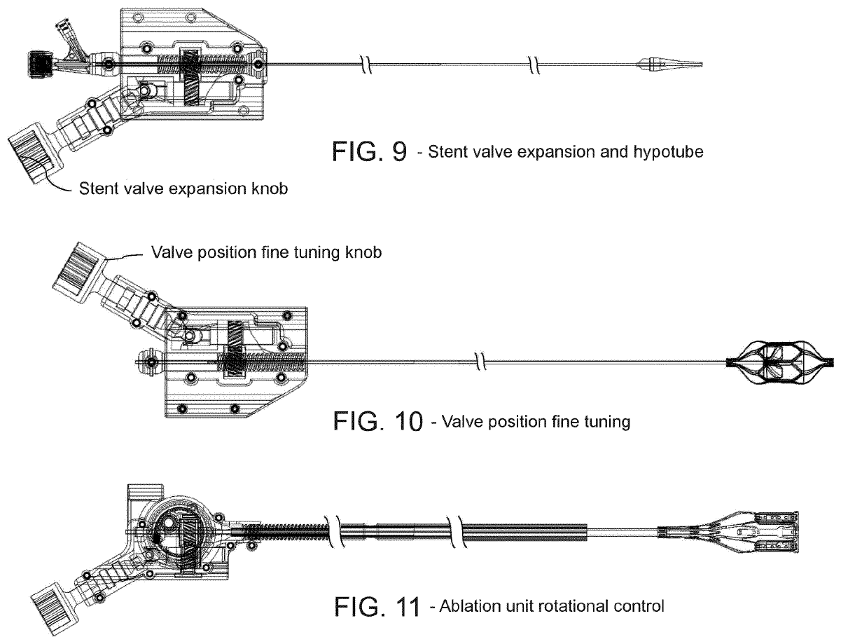

In the stent valve expansion and valve positioning fine tuning sub-units, the cardan joint transmits motion to the lead screw via two gears (FIG. 9, FIG. 10).

The steering function of the outer catheter, usually used to cross the aortic arch and reach the aortic valve, takes place via tie wires that run in the bi-lumen tube (FIG. 13). Through the “Distal section steering” (FIG. 7a), the knob, by rotating, pulls cables, made of kevlar or other high-strength material (i.e., high grade polyester) which bend the distal section.

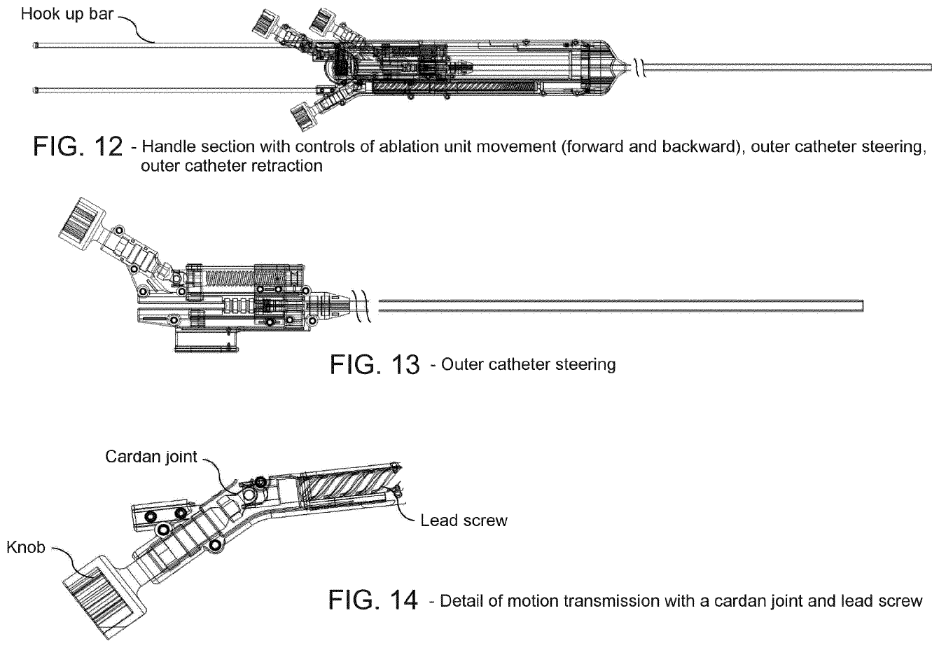

The retraction of the outer catheter (FIG. 8, FIG. 12), takes place via the lead screw to which it is fixed.

The expandable valve stent has the “catheter coupling ends” constrained to two different tubes which can slide into each other (FIG. 8b). The substructure of FIG. 8 shows the knob control to draw one tube and keep the other fixed, resulting in the expansion of the stent for compression (FIG. 3).

Two sections of the handle, one including the commands “Valve position fine tuning”, “Stent valve expansion”, “Ablation unit rotation and flexion/extension” and the other one the commands of “Ablation unit position control (forward and backward movements)”, “Distal section steering outer catheter retraction”, are assembled together via hook up bars (FIG. 7a, FIG. 12)

Inside the handle, to reduce E.M.I, electromagnetic emissions, there are 4 ferrite beads, in which the multi-lumen runs (FIG. 7b).

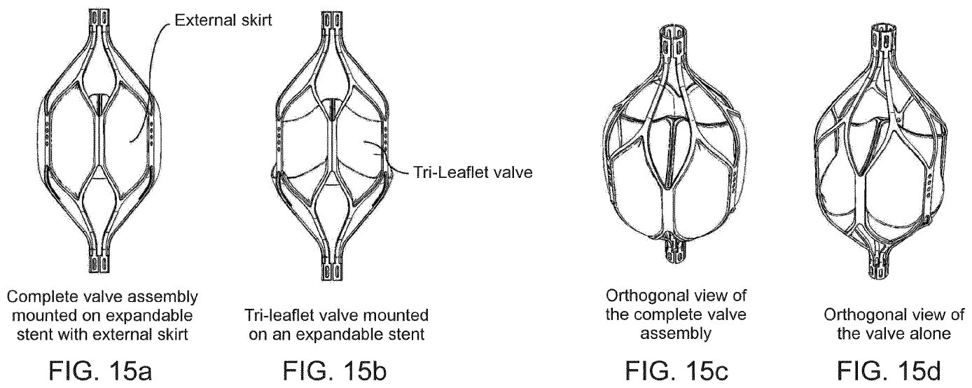

In the example of FIG. 15, the valve assembly is provided with an external skirt.

The external skirt enhances adherence to the patient's native valve, minimizing or eliminating paravalvular leakage, even with irregularities at the valve site (annulus, leaflets). The stent's upper opening ensures ample blood flow, preventing obstruction of the coronary ostia. The valve's concave belly is designed to deform flexibly, maintaining ease of opening during the systolic phase. Additionally, the apex of the coaptation zone is elevated above the rest of the leaflet's free margin. This elevation optimizes valve function by maintaining leaflet coaptation during stent expansion.

1. Device for the treatment of aortic stenosis comprising at least one ultrasonic ablation unit and one expandable stent valve; the ablation unit being configured to be moved relative to the expandable stent valve.

2. Device according to claim 1 wherein the ablation unit is configured to rotate relative the expandable stent valve.

3. Device according to claim 1 wherein the ablation unit is configured to directly act on an aortic valve leaflet.

4. Device according to claim 3 wherein the expandable stent valve is configured to clamp an aortic valve leaflet between said stent valve and the ablation unit.

5. Device according to claim 1 comprising several ablation units.

6. Device according to claim 1 wherein said stent valve comprises an external skirt.

7. Device according to claim 1 comprising the following elements:
A handle
A multi lumen catheter
At least one ablation unit
An expandable stent valve
A tip;
wherein the ablation unit being configured to be moved relative to the expandable stent valve.

8. Device according to claim 1 configured to expand the said stent valve during the treatment provided by the ablation unit(s).
